(19) United States
(12) Patent Application Publication
(10) Pub. No.: US 2023/0173154 A1
(43) Pub. Date: Jun. 8, 2023

(54) DIALYSIS APPARATUS HAVING AN APPARATUS FOR DETERMINING AT LEAST TWO HEMODIALYSIS PARAMETERS

(71) Applicant: FRESENIUS MEDICAL CARE DEUTSCHLAND GMBH, Bad Homburg (DE)

(72) Inventor: Andreas MAIERHOFER, Schweinfurt (DE)

(73) Assignee: FRESENIUS MEDICAL CARE DEUTSCHLAND GMBH, Bad Homburg (DE)

(21) Appl. No.: 17/920,045

(22) PCT Filed: Apr. 21, 2021
(86) PCT No.: PCT/EP2021/060306

§ 371 Date: Oct. 20, 2022

(30) Foreign Application Priority Data
Apr. 27, 2020 (DE) 10 2020 111 358.7

(51) Int. Cl. A61M 1/16

(52) U.S. Cl. CPC A61M 1/1617

(57) ABSTRACT

The invention relates to a dialysis device comprising a device for determining at least one first and second haemodialysis parameter, and a method for determining at least one first and second haemodialysis parameter during a dialysis treatment with a dialysis device. The device 20 for determining at least one first and second haemodialysis parameter comprises a device 21 for generating a change in a physical or chemical parameter of the dialysis fluid in the dialysis fluid system 6 in the form of a bolus, and a device 22 for detecting a change over time in the physical or chemical parameter of the dialysis fluid in the dialysis fluid system 6. Furthermore, the dialysis device has a computing and evaluating unit 23 that is configured such that, on the basis of the change in the physical or chemical parameter in the form of a bolus, for at least one dialysis condition at least one value for the clearance of the dialysis treatment is determined, and, on the basis of at least one value for the clearance, a first and second haemodialysis parameter is determined. The dialysis device according to the invention and the method according to the invention are characterised in that boluses of different sizes, in particular boluses having different area contents, are administered to determine the haemodialysis parameter.

The invention relates to a dialysis device comprising a device for determining at least one first and second haemodialysis parameter, and a method for determining at least one first and second haemodialysis parameter during a dialysis treatment with a dialysis device. Furthermore, the invention relates to a dialysis system comprising a dialysis device that comprises a dialyser and an extracorporeal blood circuit, and a device for determining at least one first and second haemodialysis parameter. The invention further relates to a computer program product comprising commands that, when the program is executed by a computer, cause the computer to carry out a method for determining at least one first and second haemodialysis parameter during a dialysis treatment with a dialysis device.

Methods for chronic blood purification therapy, such as haemodialysis, haemofiltration and haemodiafiltration, involve guiding blood through a blood treatment unit, for example a dialyser or filter, via an extracorporeal blood circuit. In the following, dialyser and filter are understood as synonyms. An arteriovenous fistula, which is generally punctured using an arterial and venous cannula, is often used to access the circulatory system. The use of a vascular implant is also possible. Whenever the term ‘vascular access’ is used below, it refers to any type of access to a patient’s circulatory system. In particular, vascular access is understood to mean the enlarged blood vessels created by the connection between an artery and a vein of the patient. The vessel may also be a vessel created by means of a graft.

Various parameters are of interest for monitoring dialysis treatment, which are referred to as haemodialysis parameters below.

A haemodialysis parameter relevant for the efficiency of dialysis is the dialysis dose Kt/V, which is a dimensionless parameter. The dialysis dose is the quotient of the product of urea clearance K (ml/min) and dialysis time t (min) and the urea distribution volume V. The clearance is the amount of blood plasma that is completely purified from a certain substance per unit of time when flowing through the kidneys or the membrane of a dialyser. ‘Dialysance’ is a common term in dialysis literature (e.g. Petitclerc, Gotch, ...) when it comes to approximating a reference concentration, e.g. sodium, i.e. if the substance under consideration is present on both sides of the membrane. The ionic dialysance may be determined online to determine the clearance of small molecules such as urea. The relationship explained below exists between the terms clearance K and dialysance D.

For medical professionals, clearance K is ‘the partial flow (in ml/min) of the blood that is completely freed of the substance under consideration’. From a mathematical point of view, however, the restriction to the case in which the transport takes place into a space in which the substance is not present is not necessary. The underlying kinetics (1-pool model) are described by V * dc b/dt = - K (c b - c d), where V: volume of distribution, dc: change in concentration, b: blood, d: dialysate; K: ‘transmembrane flow’, ‘reaction kinetics’, ...; and is directly dependent on the membrane properties and the diffusion constant of the substance under consideration, where the units [K] = ml/min, [c] = number/ml, V: volume of distribution. If c_{d}= 0, the term clearance would be used. For c_{d} > 0, K still describes the kinetics correctly, but the clear definition is then ‘partial flow that is brought completely to the concentration of the dialysate’. The term ‘dialysance’ is used for this. In the field of dialysis, the term ‘clearance’ is usually assigned to substances that are not present in the dialysate (for example urea, creatinine, beta-2-M, antibiotics) and ‘dialysance’ substances that are also present in the dialysate (for example Na). In terms of numbers, sodium dialysance would always be equal to sodium clearance, the sodium clearance being used when dialysing against RO water. For the purpose of this description, clearance K and dialysance D are to be understood as synonyms.

Various prior art methods are known for determining the clearance K (dialysance). The measurement of ionic dialysance with so-called Online Clearance Monitoring OCM (Fresenius Medical Care), which allows the indirect determination of urea clearance during dialysis, is widespread. Since the treatment duration (t) is known, the system can determine the dialysis dose Kt/V online during dialysis after the urea distribution volume (V) is entered.

The online determination of the clearance is based on the change over time of a physical or chemical parameter of the dialysis fluid in the dialysis fluid system. A physical or chemical parameter of the dialysis fluid is changed upstream of the dialyser, and the change over time of the physical or chemical parameter of the dialysis fluid downstream of the dialyser due to the change over time in the physical or chemical parameter of the dialysis fluid upstream of the dialyser is recorded. The change over time in the physical or chemical parameter of the dialysis fluid in the dialysis fluid system is referred to below as a bolus.

EP 0 911 043 B1 describes a dialysis device that has a computing and evaluating unit that is designed such that the dialysance D is determined from the amount of a substance added upstream of the dialyser as a bolus of the dialysis fluid and from the integral over the time of the change in the concentration of a substance in the dialysis fluid downstream of the dialyser attributable to the bolus as well as from the dialysis fluid flow. A bolus can be administered, for example, by changing the sodium ion concentration, for example, a solution containing NaCl can be added. Dosing can be done online with a variation of the dosing volume of a concentrate. The change can be made by adding more or less concentrate (compared to the base dosage that leads to the baseline). This allows for changes in both directions in the form of a positive bolus or a negative bolus. It should be noted here that no pure NaCl solution needs to be added as a concentrate, but a solution of different ions can also be added. When generating the bolus, it is generally only the conductivity that matters, i.e., the form of the bolus, which can be described by the area and/or amplitude and/or duration of the bolus, is influenced by the change in concentration of all ions contained in the dialysate.

Another haemodialysis parameter that is important to dialysis treatment is the blood access flow, which is referred to below as blood flow in the vascular access Qa. There are various prior art methods for determining Qa. Methods for determining Qa are known that are based on determining the clearance given different blood flow directions.

EP 0 928 614 B1 describes a dialysis device that has a device for detecting a change over time in the physical or chemical parameter of the dialysis fluid in the dialysis fluid system attributable to the bolus for two different dialysis conditions. Under the first dialysis condition, the first bloodline conveys blood from a downstream portion of a patient’s vascular access to the dialyser and the second bloodline conveys blood from the dialyser towards an upstream portion of the vascular access, and under the second dialysis condition, the first bloodline conveys blood from an upstream portion of the vascular access and the second bloodline conveys blood towards a downstream portion of the vascular access. The known method for determining Qa is based on determining a value for the dialysance for the first dialysis condition and a value for the dialysance for the second dialysis condition and calculating the blood flow in the vascular access Qa from the two values for the dialysance. The clearance (dialysance) can be determined online for the different dialysis conditions using the known methods (Online Clearance Monitoring OCM).

The object of the invention is to determine at least two haemodialysis parameter during a dialysis treatment having the greatest possible accuracy with the least possible burden on the patient.

This object is achieved according to the invention with the features of the independent claims. The dependent claims relate to preferred embodiments of the invention.

The dialysis device according to the invention has a device for determining at least one first and second haemodialysis parameter. The dialysis device is configured to be connected to a dialyser that is divided by a semi-permeable membrane into a first compartment, which is part of an extracorporeal blood circuit, and a second compartment, which is part of a dialysis fluid system. The dialysis device is further configured to be connected to a first and a second bloodline, said bloodlines being connected to an inlet or outlet of the first compartment of the dialyser. The bloodlines (blood tubing system) are part of the extracorporeal blood circuit.

The device for determining haemodialysis parameters comprises a device for generating a change over time in a physical or chemical parameter of dialysis fluid located in the dialysis fluid system in a dialysis fluid feed line to the dialyser in the form of a bolus, and a device for detecting a change over time in the physical or chemical parameter of the dialysis fluid in a dialysis fluid discharge line from the dialyser that is attributable to the bolus. The change in the physical or chemical parameter can be measured upstream of the second compartment of the dialyser, and the change in the parameter attributable to the change in the parameter can be measured downstream of the second compartment of the dialyser.

The change in the parameter can occur for a first dialysis condition under which the first bloodline conveys blood from a downstream portion of a patient’s vascular access and the second bloodline conveys blood towards an upstream portion of the vascular access and/or for a second dialysis condition under which the first bloodline conveys blood from an upstream portion of the vascular access and the second bloodline conveys blood towards a downstream portion of the vascular access.

Physical or chemical parameters can be different properties of the dialysis fluid that can be measured in the dialysis fluid system. These properties can be measured directly or variables correlating with these properties can be measured. For example, the parameter can be the concentration of a substance in the dialysis fluid (substance concentration), for example the concentration of one or more electrolytes, in particular the Na concentration. For example, the conductivity or optical properties of the dialysis fluid, for example the absorbance or the angle of rotation of the light (glucose) or the ultrasound propagation speed can be measured. Alternatively, chemical sensors can also be used for the measurement.

Furthermore, the dialysis device according to the invention has a computing and evaluating unit that cooperates with the device for generating a change in a physical or chemical parameter and the device for recording a change over time in a physical or chemical parameter, which computing and evaluating unit is configured such that, on the basis of the change in the physical or chemical parameter in the form of a bolus, at least one value for the clearance of the dialysis treatment is determined, and at least one haemodialysis parameter is determined on the basis of the at least one value for the clearance.

The inventor has recognised that the type of change over time in the parameter can be decisive for determining individual haemodialysis parameters, i.e., the form of the bolus, on the one hand from the point of view of a measurement that is as accurate as possible and on the other hand taking into account the lowest possible burden for the patient. In particular, it has been found that a bolus administered for a clearance measurement to determine the dialysis dose Kt/V is not equally suitable for a clearance measurement to determine the blood flow in the vascular access Qb with sufficient accuracy.

The dialysis device according to the invention and the method according to the invention also take into account that practice has shown that various boundary conditions must be observed to determine haemodialysis parameters, in particular to determine the dialysis dose Kt/V or the blood flow in the vascular access Qa.

The dialysis machine according to the invention and the method according to the invention are characterised in that different boluses are administered for the different measurements. A ‘large bolus’ is administered to determine the first haemodialysis parameter, while a ‘small bolus’ is administered to determine the second haemodialysis parameter. A small bolus basically has the advantage of a lower burden on the patient, while a large bolus basically has the advantage of a high measuring accuracy.

The device for generating a change in a physical or chemical characteristic value is designed
such that, to determine the first haemodialysis parameter, at least one bolus is generated having a first size (A), the computing and evaluating unit being configured such that, on the basis of the change in the physical or chemical parameter in the form of at least one bolus for determining the first haemodialysis parameter, at least one value for the clearance of the dialysis treatment is determined, on the basis of which value or values the first haemodialysis parameter is determined, and
that, to determine a second haemodialysis parameter, at least one bolus for determining the second haemodialysis parameter is generated having a second size (A′), the computing and evaluation unit being configured such that, on the basis of the change in the physical or chemical parameter in the form of at least one bolus for determining the second haemodialysis parameter, at least one value for the clearance of the dialysis treatment is determined, on the basis of which value or values the second haemodialysis parameter is determined.

A first aspect of the invention provides that the first size (A) is larger than the second size (A′), while a second aspect provides that the distance between the first size (A) and a reference value is greater than the distance between the second size (A′) and a reference value.

The size of the bolus is determined by its form. An integral (area) or the amplitude and/or duration of the bolus may be characteristic of the form of the bolus. The temporal course of the change does not need to be described precisely, but may also be approximated.

The quotient between the first area (A) and the second area (A′) is in particular greater than 2.5, in particular greater than 2.7, in particular greater than 3.

An embodiment according to the first aspect of the invention provides that the at least one bolus for determining the first haemodialysis parameter is characterised by a first area and the at least one bolus for determining the second haemodialysis parameter is characterised by a second area, the area content of the first area (first area content) is greater than the area content of the second area (second area content). Consequently, the first haemodialysis parameter can in principle be determined with a higher degree of accuracy using a ‘large bolus’, but this could cause the determination of this parameter to be associated with a higher burden on the patient. The second haemodialysis parameter can in principle be determined only with a lower accuracy using a ‘small bolus’, but a possible burden on the patient is excluded. In this context, the area content of the bolus is understood to mean the area content of the area between the graph of a function describing the change over time in the physical or chemical parameter and the graph of a reference function.

The reference function may be a baseline that can be determined by a variation of the physical or chemical parameter before and/or after the bolus. The baseline may be a straight line, a curve, or some other function other than a straight line. The straight baseline may have no slope or may have a slope. The baseline can describe the variation of the parameter adjusted for noise signals or fluctuations.

The measurement accuracy can in principle also be improved by taking several measurements and statistically evaluating the measurement results. However, this approach is associated with the disadvantage that it takes a relatively long time to determine the haemodialysis parameter and a greater burden could be placed on the patient because of the multiple measurements.

According to the second aspect of the invention, a reference value is defined, the size of the at least one bolus for determining the first haemodialysis parameter and the size of the at least one bolus for determining the second haemodialysis parameter being related to the reference value. The reference value may be the noise in the system, and the distance from the reference value may be a signal-to-noise ratio. For an accurate measurement, the largest possible signal-to-noise ratio is sought, which increases with the size of the bolus, but which also increases the burden on the patient. In the second aspect of the invention, in analogy to the first aspect, a larger signal-to-noise ratio is sought for determining the first haemodialysis parameter than for determining the second haemodialysis parameter.

The advantages of the invention are particularly evident when the first haemodialysis parameter is the blood flow in the vascular access Qb or a recirculation flow in a vascular system and the second haemodialysis parameter is the dialysis dose Kt/V.

To determine the blood flow in the vascular access, the device for generating a change in a physical or chemical parameter may be designed such that, for a first dialysis condition, a first bolus having the first area content (A) and, for the second dialysis condition, a second bolus having the first area content (A) is generated, the computing and evaluating unit being configured such that, on the basis of the change in the physical or chemical parameter in the form of the first bolus having the first area content (A) under the first dialysis condition, a first value for the clearance is determined and, on the basis of the change in the physical or chemical parameter in the form of the second bolus under the second dialysis condition, a second value for the clearance is determined, on the basis of which values the blood flow in the vascular access is determined.

Under the first dialysis condition, the first bloodline conveys blood from a downstream portion of a patient’s vascular access and the second bloodline conveys blood towards an upstream portion of the vascular access, and under the second dialysis condition, the first bloodline conveys blood from an upstream portion of the vascular access and the second bloodline conveys blood towards a downstream portion of the vascular access.

In other words, the dialysis condition comprises the needle orientation, which can be designated as a normal orientation or inverted orientation, for example. Changing the needle orientation or the first and second dialysis conditions also means that the needles are exchanged or the flow through the needles is reversed. This can be achieved, for example, by the blood pump pumping in the opposite direction or by a flow reversing device being provided, by means of which the needle that is venous during normal treatment is connected to the arterial blood tube (or a portion thereof) and the needle that is arterial during normal treatment is connected to the venous blood tube (or a portion thereof). This flow reversing device can be operated manually or controlled automatically. An example of a manually operated flow reversing device is the so-called Twister® from Fresenius Medical Care Deutschland GmbH.

For the two dialysis conditions, all other parameters that have an influence on the clearance are preferably the same or largely the same during the measurements. If said other parameters are not the same, the dialysis device may be designed such that when determining the blood flow in the vascular access or a recirculation flow in a vascular system, the different parameters are computationally corrected for the influence of these differences at certain clearance values. For example, an increased dialysis fluid flow can be corrected by means of a stored dependence of the clearance on the dialysis fluid flow. This dependency can be stored in the form of a table or an equation or algorithm.

An alternative embodiment provides that the dialysis device is designed such that, under the first dialysis condition, a clearance value is not measured but estimated, and, under the second dialysis condition, a clearance value is measured by means of the method described above while changing a physical or chemical parameter. The blood flow in the vascular access Qa is then determined using the estimated clearance value and the measured clearance value.

The estimation of the clearance instead of a measurement is generally of sufficient accuracy only for determining the first dialysis parameter under the first dialysis condition, in particular the blood flow in the vascular access, because fistula recirculation is not to be expected or is at least low under the first dialysis condition. Under the second dialysis condition, however, fistula recirculation does occur. Therefore, an estimation of the second dialysis parameter makes no sense under the second dialysis condition.

The alternative embodiment comprising an estimate of the clearance instead of a measurement for determining the first clearance value under the first dialysis condition to determine the first dialysis parameter, in particular the blood flow in the vascular access, is of its own inventive importance, i.e., determining the first dialysis parameter using this method can take place independently of the determination of a second dialysis parameter and independently of the principle according to the invention of dimensioning boluses of different sizes.

In the alternative embodiment, to determine the blood flow in the vascular access Qa, the device for generating a change in a physical or chemical parameter is designed in such way that a bolus having the first area content (A) is generated for a second dialysis condition, the computing and evaluating unit being configured such that, on the basis of an estimate of the clearance for the first dialysis condition, a first value for the clearance is determined and, on the basis of the change in the physical or chemical parameter in the form of the bolus having the first area content (A) under the second dialysis condition, a second value for the clearance is determined, on the basis of which values the blood flow in the vascular access is determined.

If the first haemodialysis parameter, in particular the blood flow in the vascular access Qa, is determined using the method described above on the basis of an estimate of the first clearance value instead of a measurement of the clearance value with a bolus, the dialysis device can optionally also be designed to only carry out individual steps for determining Qa if certain conditions are met that indicate sufficient accuracy.

The dialysis device can be configured such that, after the first clearance value has been estimated, the second clearance value is only measured if a predetermined condition for the estimation of the first clearance value is met, for example if the estimated clearance value does not meet certain plausibility criteria, for example the deviation of the estimated clearance value from a specified value is too great.

If the deviation is too great, the first clearance value can be measured after administering a bolus in order to have a value with sufficient accuracy. The second clearance value may then be measured.

Instead of checking the estimated first clearance value, the blood flow in the vascular access Qa determined from the first and second value can also be checked after estimating the first clearance value and measuring the second clearance value. If the blood flow in the vascular access Qa does not make sense or deviates too much from an expected blood flow value in the vascular access, the blood flow can be determined on the basis of two successive measurements using a first and a second bolus. For this purpose, the dialysis device may be designed such that the first clearance value is then also measured.

The clearance can be estimated, for example, using the equations published in Sargent, A. & Gotch, F., ‘Principles and biophysics of Dialysis’ in ‘Replacement of renewal function by dialysis’, 4th ED. To determine an estimated value for the clearance, certain parameters of the dialyser can be determined in the laboratory or manufacturer information, for example in the form of a code (bar code) present on the dialyser or a printed number to be transmitted by the user, or parameters stored in the dialysis machine for the user by selecting a dialyser type stored parameters, can be used. The blood flow, dialysate flow, ultrafiltration flow across the dialyser membrane and/or substituate flow can also be taken into account. The equation used for the clearance or dialysance can be (without ultrafiltration flow or substituate flow):

$$D = {Q}_{b} \frac{{e}^{γ} - 1}{{e}^{γ} - \frac{{Q}_{b}}{{Q}_{d}}} , γ = {k}_{0} A \frac{{Q}_{d} - {Q}_{b}}{{Q}_{b} {Q}_{d}}$$

kₒA mass transfer coefficient membrane surface product

Q_{b} is the blood flow through the dialyser.

Q_{d} is the efficient dialysate flow flowing through the dialyser.

The clearance can also be estimated using the method described in EP 1 698 360 B1. The following method for estimating the clearance is disclosed in EP 1 698 360 B1:

The clearance K, which changes as a function of time during therapy, is calculated using the following equation:

$$\text{K} \left(\text{t}\right) = {\text{K}}_{\text{L}} * \text{C} \left(\text{t}\right)$$

K is the clearance value that changes with the therapy time t.

K_{L} is the clearance of the type of dialyser in question determined under laboratory conditions.

C(t) is a correction factor that describes the reduction in the filter efficiency K_{L} over the therapy time, for example, by forming a secondary membrane.

C is less than or equal to 1, C(t) = 1-(X₁ * t), where X₁ is an empirically determined patient/filter factor.

K_{L} is a function of a plurality of device parameters:

$${\text{K}}_{\text{L}} = \text{f} \left({\text{Q}}_{\text{b}} ; {\text{Q}}_{\text{d}} ; {\text{Q}}_{\text{u}} ; \text{FT}\right)$$

Q_{b} is the blood flow flowing through the dialyser.

Q_{d} is the efficient dialysate flow flowing through the dialyser.

Qᵤ is the ultrafiltration flow flowing across the membrane.

FT is the filter type.

K_{L} is usually determined from tables or three-dimensional matrices, the values of which were measured under laboratory conditions, and which are specified in the instructions for use for the various dialysers.

If the first clearance value is estimated or measured under the first dialysis condition and the second clearance value is measured under the second dialysis condition, the first dialysis parameter, in particular the blood flow in the vascular access Qa, can be calculated from the first and second clearance values.

The blood flow in the vascular access Qa can be calculated from the equation (4) specified in the description of the figures below.

Alternatively, the blood flow in the vascular access Qa can be determined using the following equation:

$$\text{Qa} = \left(\text{1/R-1}\right) \text{*Qb}$$

Qa is the blood flow in the vascular access

R is the fistula recirculation (with the needle position reversed)

Qb is the blood flow through the dialyser.

To determine the blood flow in the vascular access Qa using the above equation, the recirculation in the vascular access (fistula recirculation) R must first be determined. The recirculation in the vascular access can also be estimated. An estimate for recirculation in the vascular access can be determined from the measured clearance and the estimated clearance under the different dialysis conditions, for example from the ratio of the measured clearance and the estimated clearance, the estimate consisting in taking the cardiopulmonary recirculation into account.

A method for determining the blood flow value in the vascular access while estimating the clearance may therefore include the following steps:
1. Determining an estimated value for the clearance based on dialyser parameters (e.g. manufacturer data, bar code, etc.);
2. Changing the dialysis condition, in particular reversing the needle orientation;
3. Measuring the clearance in the changed dialysis condition or in the swapped needle position;
4. Determining the blood flow value in the vascular access Qa using the estimated clearance and the measured clearance, it being possible for step 4 to comprise steps 4a1. and 4b1. or step 4a2.;
4a1. Determining an estimate for recirculation in the vascular access from the measured clearance and the estimated clearance, for example from the ratio of the measured clearance and the estimated clearance, the estimate consisting in taking the cardiopulmonary recirculation into account. The clearance estimated using, for example, the estimation methods described above may be too high due to the cardiopulmonary recirculation. To determine the recirculation in the vascular access, the cardiopulmonary recirculation can therefore be estimated, for example by using a customary value or a value previously determined for the patient, thus reducing the estimated clearance. For example, in a first approximation it can be assumed that the total recirculation is the sum of the cardiopulmonary recirculation and the recirculation in the vascular access, and, accordingly, the estimated clearance is reduced by the proportion of the cardiopulmonary recirculation. In another approximation, the total recirculation can be estimated from the estimated clearance and the measured clearance, and the cardiopulmonary recirculation can then be subtracted from the total recirculation to determine the recirculation in the vascular access;
4b1. Determining the blood flow value in the vascular access, for example using the formula Qa = (1/R-1)*Qb;
4a2. Determining the blood flow value in the vascular access using equation (4), and optionally determining the recirculation using the formula Qa = (1/R-1)*Qb solved for R.

The accuracy of the paths via 4a1 or 4a2 depends on the accuracy of the estimates. The estimated clearance can be calculated from the manufacturer’s information and the flows. Then, the estimated clearance is overestimated because the reduction due to cardiopulmonary recirculation is not taken into account. When taking the path via recirculation (step 4a.1), overestimating the estimated clearance leads to high values for the recirculation and thus to low values for Qa (step 4b.1). Directly entering the estimated clearance into equation (4) leads in the same direction (step 4a2.). When estimating the estimated clearance from the manufacturer’s information, a typical cardiopulmonary recirculation value (e.g. 5%, 10%, or 15%) can therefore be used to better estimate the estimated clearance.

To determine the blood flow in the vascular access, the device for generating a change in a physical or chemical parameter can be designed such that the substance concentration of a substance in the dialysis fluid, in particular the Na concentration, is increased under the first and second conditions for determining the first and second value of the clearance or decreased under the first and second dialysis conditions for determining the first and second values of the clearance. If there is a change in ‘a substance, in particular the Na concentration’, this always means that corresponding counterion concentrations must also be changed in the case of ions. In fact, there is no elemental sodium in the solution, for example, but positively charged sodium ions. The corresponding negative counterions may be chloride ions. As a rule, the negative counterions are chloride ions. Other counterions in a conventional dialysis fluid are carbonate or bicarbonate ions. In other words, changing the concentration of a substance involves changing the concentration of one or more types of uncharged molecules, and changing the concentration of an ion and a counterion involves changing the concentration of a plurality of ion and counterion types.

The inventor has recognised that a bolus sufficient in size for determining the dialysis dose Kt/V is insufficient for determining the blood flow in the vascular access Qa with sufficient accuracy and reliability. The reason for this seems to be that determining the blood flow in the vascular access Qa requires two values, with two values being entered into the equation to calculate Qa such that measurement errors can have a much greater impact than if a haemodialysis parameter is determined merely on the basis of a measurement.

An embodiment of the dialysis device according to the invention provides a controlling and computing unit that cooperates with the device for generating a change in a physical or chemical parameter and the device for measuring a change over time in a physical or chemical parameter, which controlling and computing unit starts the generation and detection of the change in the physical or chemical parameter during the blood treatment to determine a haemodialysis parameter. The controlling and computing unit can automatically change and record the parameter during the blood treatment while taking into account certain time requirements, it being possible to store the time for a one-time or two-time determination of a dialysis parameter or the times for a multiple determination of a dialysis parameter in a memory of the controlling and computing unit. However, the time or the times can also be entered by the medical personnel via an input unit and read into the memory. The controlling and computing unit can also carry out the necessary evaluations of the measurement signals and perform the calculations or can be programmed for this purpose.

The controlling and computing unit may be designed such that, to determine the first haemodialysis parameter, in particular the blood flow in the vascular access Qa, the generation and detection of the change in the physical or chemical parameter is started only once or only twice during the blood treatment. A one-time determination of Qa is generally sufficient. It has been shown that a high measuring accuracy can be achieved for the determination of Qa using the larger bolus. In practice, a scatter of around 20% is tolerable for the measurement of Qa to allow a trend analysis with typical blood flows of approx. 1200 ml/min, which can require reproducibility of the clearance measurement of 2% regardless of the needle orientation. Because Qa is only measured once per dialysis treatment, a larger bolus can be tolerated.

The controlling and computing unit may be designed such that, to determine the second haemodialysis parameter, the generation and detection of the change in the physical or chemical parameter is started several times during the blood treatment, it being possible for time requirements to be made automatically or by medical personnel. It has been shown that a sufficient measurement accuracy can be achieved for the determination of the dialysis dose Kt/V using a bolus that is smaller than the bolus for the determination of the blood flow in the vascular access Qb. Because it is not possible for a small bolus to be a burden on the patient, several measurements can be taken during the blood treatment without any problems.

The device for generating a change in a physical or chemical parameter may be designed for determining the first haemodialysis parameter such that the substance concentration of a substance in the dialysis fluid, in particular the Na concentration, is increased for determining the first and second value of the clearance under the first and the second dialysis conditions or decreased for determining the first and second values of the clearance under the first and second dialysis conditions. Determining the first haemodialysis parameter using two ‘negative’ boluses has the advantage that the substance concentration in the dialysis fluid, in particular the Na concentration, is not increased. Increasing the Na concentration can lead to an increase in the patient’s blood pressure. Determining the first haemodialysis parameter using two ‘positive’ boluses is associated with a lower risk of intradialytic drops in blood pressure and cramping that can be caused by a reduction in the Na concentration.

If several measurements are to be carried out to determine the second dialysis parameter, the device for generating a change in a physical or chemical parameter may be designed such that the substance concentration of a substance in the dialysis fluid, in particular the Na concentration, increases for a preceding measurement and decreases for a subsequent measurement or decreases for a preceding measurement and increases for a subsequent measurement. The alternating administration of a ‘positive’ and a ‘negative’ bolus has the advantage of a smaller change in the substance concentration in the dialysis fluid, in particular the Na concentration.

The invention further relates to a computer program product that comprises commands that, when the program is executed by a computer, cause the computer to carry out the methods described above for determining at least one first and second haemodialysis parameter during a dialysis treatment with a dialysis device.

In the following, an embodiment of the invention will be described in detail making reference to the drawings.

In which:

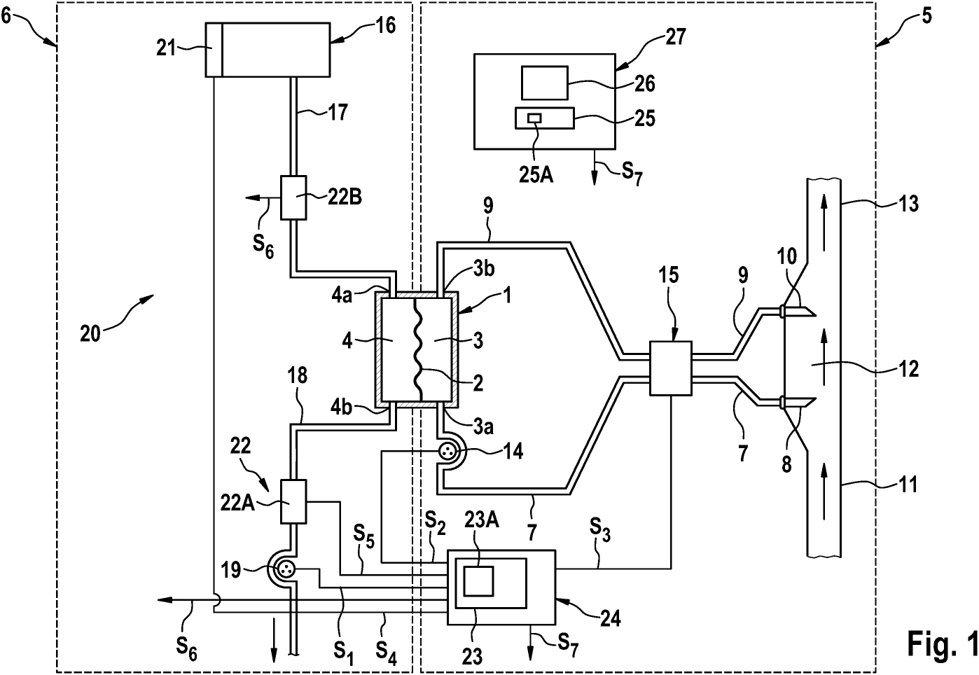

FIG. 1 is a simplified schematic view of an embodiment of the dialysis device according to the invention,

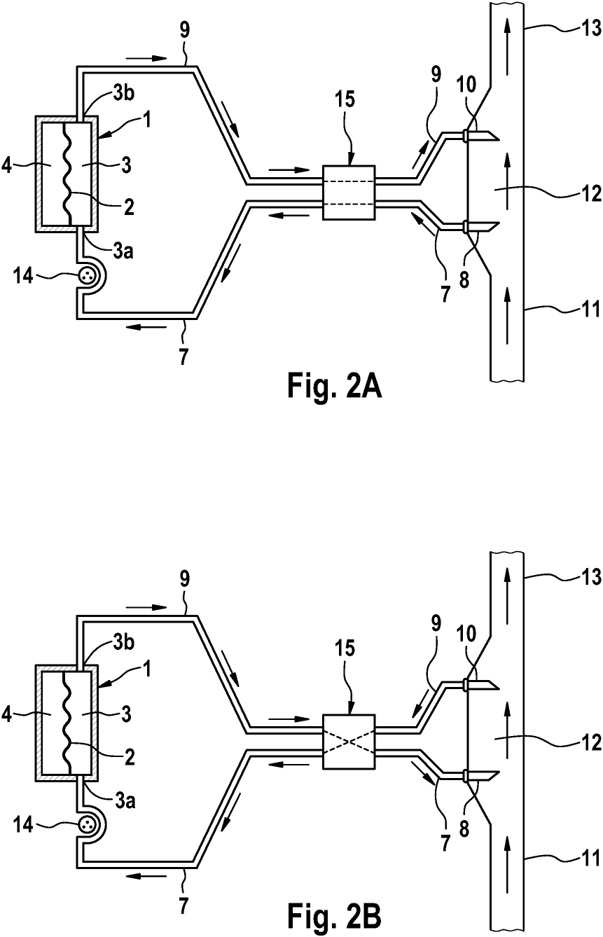

FIG. 2A shows the device for specifying a dialysis condition of the dialysis device, a first dialysis condition being specified,

FIG. 2B shows the device for specifying a dialysis condition of the dialysis device, a second dialysis condition being specified,

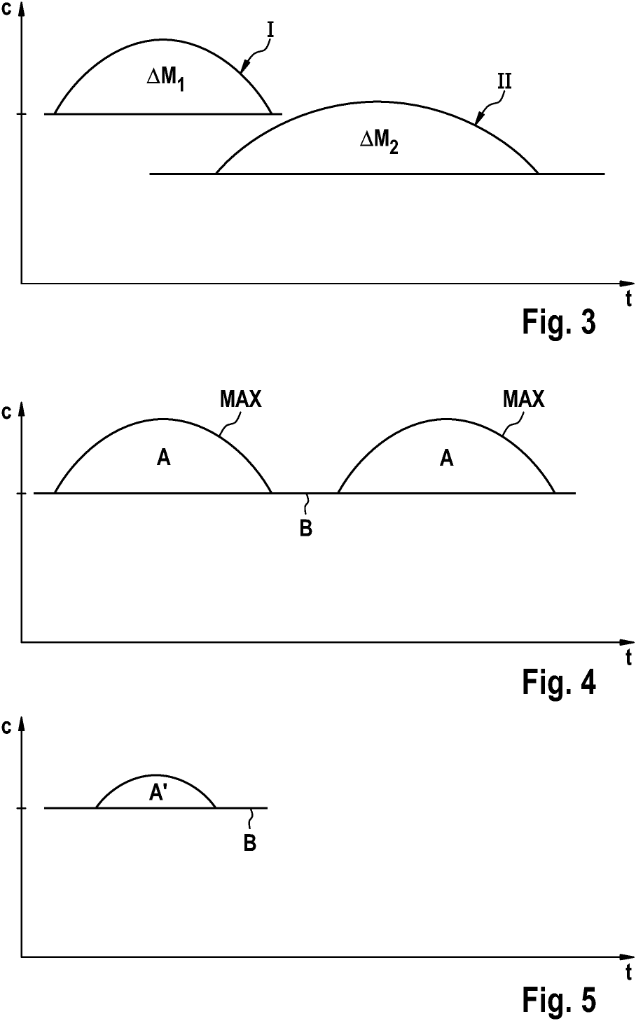

FIG. 3 shows the change over time in the physical or chemical parameter of the dialysis fluid at the inlet and outlet of the dialyser,

FIG. 4 shows the first and second change over time in the physical or chemical parameter of the dialysis fluid at the inlet of the dialyser for determining a first haemodialysis parameter, a positive bolus being administered,

FIG. 5 shows the change over time in the physical or chemical parameter of the dialysis fluid at the inlet of the dialyser for determining a second haemodialysis parameter,

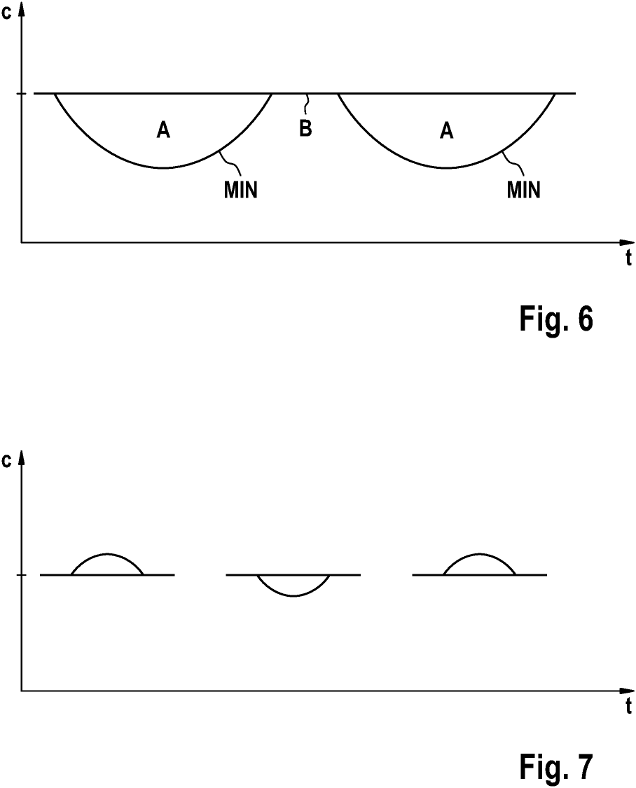

FIG. 6 shows the first and second changes over time in the physical or chemical parameter of the dialysis fluid at the inlet of the dialyser for determining a first haemodialysis parameter, a negative bolus being administered,

FIG. 7 shows a sequence of positive and negative boluses for continuously determining the second haemodialysis parameter during the blood treatment,

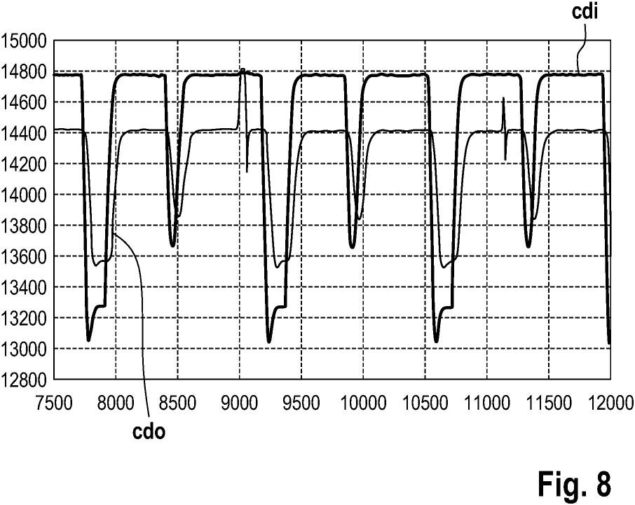

FIG. 8 shows the conductivity of the dialysis liquid measured in an experiment upstream and downstream of the dialyser.

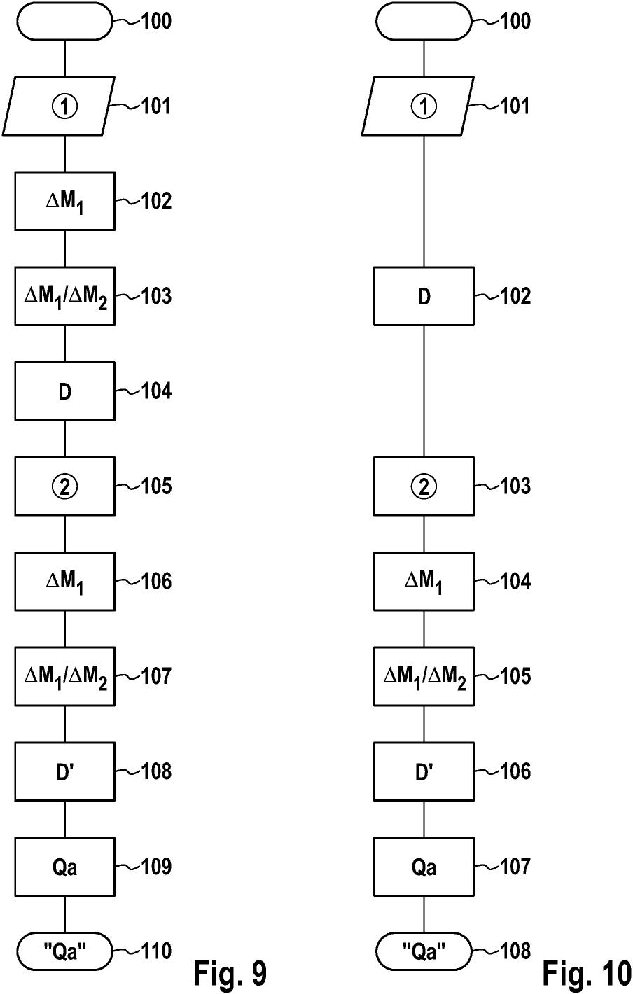

FIG. 9 shows a flow chart to illustrate the determination of a first dialysis parameter on the basis of a first and second measurement of a first and second clearance value under a first and second dialysis condition,

FIG. 10 shows a flow chart to illustrate the determination of a first dialysis parameter on the basis of an estimate of a first clearance value and a measurement of a second clearance value under a first and second dialysis condition.

FIG. 1 is a simplified schematic representation of the components of an embodiment of the dialysis device according to the invention that are essential to the description of the invention. Because the measurement methods for determining the haemodialysis parameters as such are known from the prior art, only those aspects of the method that are essential to the invention are described.

While operating, the dialysis device is connected to a dialyser and an extracorporeal blood tubing system. The dialysis device is programmed to be able to carry out the method according to the invention to determine the dialysis parameters if it is connected both to a dialyser and an extracorporeal blood tubing system and to a patient.

The dialysis device according to the invention may be equipped with a dialyser 1 that is divided by a semi-permeable membrane 2 into a first compartment 3 and a second compartment 4. The first compartment 3 of the dialyser 1 is part of an extracorporeal blood circuit 5 shown in dotted lines in FIG. 1, and the second compartment 4 of the dialyser 1 is part of a dialysis fluid system 6 shown in dotted lines.

The extracorporeal blood circuit 5 comprises a first bloodline 7, at one end of which a first cannula 8 is connected, and a second bloodline 9, at the one end of which a second cannula 10 is connected. The first cannula 8 is connected to a downstream portion 11 of a patient’s vascular access 12, and the second cannula 10 is connected to an upstream portion 13 of the patient’s vascular access 12. The direction of the blood flow in the vascular access is designated by an arrow. The other end of the first bloodline 7 is connected to an inlet 3a of the first compartment 3 and the other end of the second bloodline 9 is connected to an outlet 3b of the first compartment 3 such that the patient’s blood flows from the downstream portion 11 of the vascular access into the first compartment 3 and flows from the first compartment 3 into the upstream portion 13 of the vessel access 12. The blood is conveyed in the extracorporeal blood circuit 5 using a blood pump 14.

The dialysis device can have a device 15 for specifying a first and a second dialysis condition that is shown in a highly simplified manner in FIGS. 1A and 1B. The device 15 for specifying a dialysis condition is designed such that a first dialysis condition can be specified under which the first bloodline 7 conveys blood from the downstream portion 11 of the vascular access 12 and the second bloodline 8 conveys blood towards the upstream portion 13 of the vascular access 12 and a second dialysis condition can be specified under which the first bloodline 7 conveys blood from the upstream portion 13 of the vascular access 12 and the second bloodline 9 conveys blood towards the downstream portion 11 of the vascular access 12. FIG. 2A shows the direction of blood flow under the first dialysis condition, and FIG. 2B shows the direction of blood flow under the second dialysis condition. The blood flow is reversed in the line portions on the vascular side.

The device 15 for specifying a dialysis condition may comprise an automatically operable arrangement of valves that allow portions of the bloodlines on the device side and on the patient side to be swapped, as shown in dotted lines in FIGS. 1A and 1B.

The device 15 may also be a manually operable device that is not connected to the dialysis device, in particular a control device of the dialysis device. The dialysis device may have an input device with which the medical personnel can input which dialysis condition is to be present. The input device can also indicate which dialysis condition was specified or that the dialysis condition was changed, for example from a first dialysis condition to a second dialysis condition. An example of such a device is the so-called Twister® from Fresenius Medical Care Deutschland GmbH.

The dialysis fluid is provided using a device 16 for providing dialysis fluid, to which a dialysis fluid feed line 17 is connected, which dialysis fluid feed line leads to an inlet 4a of the second compartment 4 of the dialyser 2. A dialysis fluid discharge line 18 is connected to the outlet 4b of the second compartment 4, which leads to a drain that is not shown. The dialysis fluid is conveyed through the second compartment 4 of the dialyser 1 in the dialysis fluid system 6 by means of a dialysis fluid pump 19.

The dialysis device has a device 20 for determining a first haemodialysis parameter and a second haemodialysis parameter. In the present embodiment, the first haemodialysis parameter is the dialysis dose Kt/V and the second haemodialysis parameter is the blood flow in the vascular access Qa (blood access flow).

The device 20 for determining haemodialysis parameters comprises a device 21 for generating a change over time in a physical or chemical parameter of the dialysis fluid in the dialysis fluid system 6 in the dialysis fluid feed line 17 upstream of the second compartment 4 and a device 22 for detecting the change over time in the parameter in the dialysis fluid discharge line 18 downstream of the second compartment 4 attributable to the change over time in the parameter upstream of the dialyser. Furthermore, the parameter can also be recorded in the dialysis fluid feed line 17 upstream of the second compartment 4 of the dialyser 1.

The change over time in the parameter upstream of the dialyser 1 is also referred to below as the input bolus and the change over time in the parameter downstream of the dialyser is referred to as the output bolus. The output bolus is the system’s response to the input bolus.

In the present embodiment, the physical or chemical parameter is the concentration c of a substance in the dialysis liquid, in particular the Na concentration, that is changed upstream of the dialyser 1, i.e., increased or decreased for a predetermined time interval. In the present embodiment, the device 21 for generating a change over time in a physical or chemical parameter is a component of the device 16 for providing the dialysis fluid, which allows the Na concentration of the dialysate to be changed for a predetermined time interval.

In the present embodiment, the device 22 for detecting the physical or chemical parameter has a conductivity sensor 22A that measures the conductivity of the dialysis fluid in the dialysis fluid discharge line 18 as a variable correlating with the Na concentration. The device 22 for detecting the physical or chemical parameter may have a further conductivity sensor 22B that measures the conductivity of the dialysis fluid in the dialysis fluid feed line 17. If the form of the input bolus is known, the conductivity sensor 22B in the dialysis fluid feed line 17 is not needed. Otherwise, the conductivity in the dialysis fluid feed line 17 can be measured using the conductivity sensor 22B.

FIG. 3 shows the input bolus and output bolus. The bolus in the present embodiment is characterised by the area content of the area, which is between the graph of a function describing the change over time in the physical or chemical parameter, for example the Na concentration c(t) or the conductivity, and the graph of a reference function. The area content is a measure of the size of the bolus, i.e., a larger bolus has a larger area content than a smaller bolus. The reference function may be a linear function: f(t) = mt+B, where m is the slope of the function and B is a predetermined base value. The slope is 1 in the present embodiment. The base value B may be the Na concentration (conductivity) of the blood before the bolus is administered. The area content of the input and output bolus can be determined by calculating the integral in a given integration interval.

Alternatively or in combination, other variables may also be used to characterise the bolus and thus to determine the haemodialysis parameters. An alternative characteristic variable for the bolus may be the amplitude and duration of the bolus. The area content can be estimated from the amplitude and duration. The amplitude and duration of the bolus alone can be meaningful variables if a relationship between the size and duration of a bolus is known.

The determination of haemodialysis parameters based on the area content of the bolus is described below. The other variables can be used in a comparable manner.

The dialysis device has a computing and evaluating unit 23 that cooperates with the device 21 for generating a change in a physical or chemical parameter and the device 22 for recording the parameter, which computing and evaluating unit may be part of a central computing and controlling unit 24 of the dialysis device. The central computing and controlling unit 24 is connected via control and signal lines S₁ to S₆ to the dialysis fluid pump 19, the blood pump 14, the device 15 for specifying the dialysis condition, the device 21 for generating a change in a physical or chemical parameter (input bolus) and the conductivity sensors 22A and 22B of the device 22 for detecting the parameter (output bolus). The computing and controlling unit 24 is configured such that the individual components of the dialysis device are controlled during the dialysis treatment such that the method steps described below for determining the haemodialysis parameters are carried out.

The individual method steps of a first embodiment for determining a first dialysis parameter are shown in FIG. 9.

The computing and controlling unit 24 may have, for example, a general processor, a digital signal processor (DSP) for continuously processing digital signals, a microprocessor, an application-specific integrated circuit (ASIC), an integrated circuit consisting of logic elements (FPGA) or other integrated circuits (IC) or hardware components to perform the individual method steps for controlling the blood treatment device. A data processing program (software) may run on the hardware components in order to carry out the method steps.

In addition, an input unit 25 is provided that is connected to the computing and controlling unit 24 via a signal line S₇. Using the input unit 25, the medical personnel can make inputs relating to the determination of the haemodialysis parameters. The input unit 25 may have a keyboard. A display unit 26 is provided for displaying the haemodialysis parameters. The input and display unit may also be designed as a touch-sensitive screen 27 (touchscreen).

At the beginning of the dialysis treatment or after a predetermined time interval has elapsed or after a button 25A has been pressed on the touchscreen 27 by the medical personnel, the blood flow in the vascular access Qa (blood access flow) is determined (step 101).

The device 15 for specifying the dialysis condition specifies the first dialysis condition (FIG. 2A) in order to determine a first value for the clearance (dialysance) (step 102). The device 21 for generating a change in a physical or chemical parameter then generates an input bolus I having a predetermined area content ΔM₁ (step 103) (FIG. 3). The device 22 for detecting a physical or chemical parameter then detects the input bolus I having the area content ΔM₁ upstream of the dialyser 1 and the output bolus II having the area content ΔM₂ downstream of the dialyser by measuring the conductivity of the dialysis fluid using the conductivity sensors 22A and 22B upstream and downstream of the dialyser 1. The measured values are stored in a memory 23A of the computing and evaluating unit 23 (step 104).

The amount of substance ΔM1 that is fed upstream of the dialyser and the amount of substance ΔM2 that is discharged downstream of the dialyser are calculated as follows:

$$Δ \text{M1=Qd*} \int \text{dcDi*dt}$$

$$Δ \text{M2=Qd*} \int \text{dcDo*dt}$$

where Qd is the dialysis fluid flow, cDi is the dialysate concentration at the inlet and cDo is the dialysate concentration at the outlet of the second compartment 4 of the dialyser 1 and t is the time.

The computing and evaluating unit 24 can calculate a first value for the dialysance D according to the following equation (step 105):

$$\text{D=Qd*} \left(Δ \text{Mi-} Δ \text{Mo}\right) \text{/} Δ \text{Mi}$$

To determine the dialysance, more complex equations can be used that take other parameters into account, such as the ultrafiltration rate (Qf) and/or substituate rate (Qs). These equations for determining dialysance have been known for a long time and are described in the specialist literature. A more complex equation of this kind may look as follows, for example: D= (Qd+Qs+Qf)*(1- ΔM2/ΔM1) and is included as embodiments in equation (3).

The dialysis fluid flow Qd is predetermined by the dialysis fluid pump 19, which is controlled by the controlling and computing unit 24 with the corresponding flow rate.

After the first value for the dialysance has been determined, the device 21 for specifying the dialysis condition specifies the second dialysis condition (step 106) (FIG. 2B) in order to be able to determine a second value for the clearance (dialysance). The device 21 for generating a change in a physical or chemical parameter then generates an input bolus having a predetermined area content (step 107). The second input bolus preferably has the same or at least approximately the same area content as the first input bolus. The conductivity sensor 22A upstream of the dialyser 1 then detects the input bolus and the conductivity meter 22B downstream of the dialyser detects the output bolus (step 108).

As already stated above, the dialysis device may also be programmed such that the input boluses are not measured directly, but can also be calculated over time when the amount of sodium added is known. The same applies if the flows and the behaviour of the dialysis fluid in the dialysis fluid system is known for other variables characterising the input boluses.

After ΔMI and ΔM2 are calculated according to equation (1) or equation (2), the computing and evaluating unit calculates the second value D′ for the dialysance according to equation (3) (step 109).

The computing and evaluating unit now calculates the blood flow in the vascular access Qa according to the following equation (step 110):

$$\text{Qa= D*D′ /} \left(\text{D} - \text{D′}\right) ,$$

where D is the first value of the dialysance and D′ is the second value of the dialysance.

More complex equations can also be used that take other parameters into account, such as the ultrafiltration rate (Qf) and/or blood water flow (bwf). These equations for determining dialysance have been known for a long time and are described in the specialist literature. A more complex equation of this kind may look as follows, for example: Qa = 1/bwf * (D-Qf)*D′/(D-D′) and is included as embodiments in equation (3).

The blood flow in the vascular access Qa is displayed on the display unit 26 (step 11).

FIG. 4 shows the input bolus for the first and second clearance measurements. The area content is designated as A in FIG. 4 and the base value is designated as B. The device 21 for generating the input bolus increases the substance concentration of the dialysis fluid cD, in particular the Na concentration, from the basic value B to a maximum value MAX, i.e., the blood flow in the vascular access Qa is determined with two positive boluses that are characterised by the same area content A.

FIG. 6 shows an alternative embodiment having a negative input bolus for the first clearance measurement and a negative input bolus for the second clearance measurement. In FIG. 6, the area content is once again labelled A and the base value B. The device 21 for generating the input bolus reduces the substance concentration of the dialysis fluid cD, in particular the Na concentration, from the basic value B to a minimum value MIN, i.e., the blood flow in the vascular access Qa is determined with two negative boluses that are characterised by the same area content A.

Before the clearance measurements for determining the first haemodialysis parameter are carried out, the dialysis fluid flow and/or the blood flow may be increased to a predetermined value by the computing and controlling unit 24 increasing the speed of the dialysis fluid pump 19 or the blood pump 14. After the clearance measurement, the dialysis fluid flow and/or the blood flow can again be reduced to the previously set value.

After the clearance measurements, the computing and controlling unit 24 can also specify an operating mode for determining the first haemodialysis parameter, which operating mode causes a reduction in the concentration of the substance administered, in particular the salt added. Measures for reducing the salt content in the dialysate over a predetermined period of time or with a predetermined profile are described, for example, in DE 3 223 051 A1.

The method for clearance measurement as such is described in EP 0 911 043 B1, and the method for determining the blood flow Qa (blood access flow) in the vascular access on the basis of two successive clearance measurements as such is described in detail in EP 0 928 614 B1.

The above-described determination of the blood flow in the vascular access is carried out automatically only once during the dialysis treatment or after the button 25A is pressed by the medical personnel.

The dialysis device according to the invention not only provides for the determination of the blood flow in the vascular access, but also the determination of the dialysis dose kt/V during the dialysis treatment.

During the dialysis treatment, the device 21 for generating a physical or chemical property generates an input bolus, preferably under the first dialysis condition, by increasing or decreasing the Na concentration of the dialysis liquid based on the basic value B.

FIG. 5 shows an input bolus A′, the Na concentration being increased. The device 22 for detecting a physical or chemical parameter then detects the input bolus upstream of the dialyser 1 and the output bolus downstream of the dialyser by measuring the conductivity of the dialysis fluid using the conductivity sensors 22A, 22B upstream and downstream of the dialyser 11. The measured values are stored in the memory 23A of the computing and evaluating unit 23, and the dialysance D (clearance K) is calculated according to equation (3). After the dialysance D (clearance K) has been determined, the computing and evaluating unit 23 calculates the dialysis dose Kt/V, which is displayed by the display unit 26.

While the first haemodialysis parameter (blood access flow) is only determined on the basis of an input bolus that has the area content A, the second haemodialysis parameter (dialysis dose) is only determined on the basis of an input bolus that has the area content A′, the area content A being at least 2.5 times as large, in particular at least 2.7 times, in particular at least 3 times as large as the area content A′ (FIG. 4, FIG. 5).

The quotient between the area content A and A′ of the input bolus for determining the first and second haemodialysis parameters can be determined as a function of the signal-to-noise ratio. The quotient can be calculated using an algorithm as a function of the signal-to-noise ratio, or the corresponding values can be stored in the memory 23A of the computing and evaluating unit 23. The memory 23A can also store values that specify a specific pulse shape and/or pulse duration for the input bolus.

While the blood flow in the vascular access Qa is determined with a relatively large bolus, the dialysis dose Kt/V is determined with a relatively small bolus. Based on the large bolus, the blood flow in the vascular access can be determined with a high degree of accuracy. In general, a one-time determination of the blood flow in the vascular access is sufficient, preferably at the beginning of the dialysis treatment. In contrast, the dialysis dose is preferably determined multiple times during the dialysis treatment.

FIG. 7 shows a sequence of input boluses for determining the dialysis dose. A positive bolus for a previous measurement is followed by a negative bolus for a subsequent measurement, a positive Na bolus being associated with an increase in the Na concentration and a negative Na bolus being associated with a decrease in the Na concentration.

Other sequences can alternatively or additionally be programmed. In the case of an alternating sequence between positive and negative boluses, every second bolus may be directed upwards, and in the case of a sequence of positive boluses, all boluses may be directed to values greater than the baseline. The dialysis machine is preferably controlled according to alternating boluses or solely with positive boluses. FIGS. 3 to 7 are for illustrative purposes only.

FIG. 8 shows the conductivity measured in an experiment upstream and downstream of the dialyser as a function of time t. The measured conductivity correlates with the concentration of the substance (Na concentration) in the dialysis fluid. In FIG. 8, the substance concentration (conductivity) upstream of the dialyser 1 (input bolus) is denoted by cdi and the substance concentration (conductivity) downstream of the dialyser (output bolus) is denoted by cdo.

Upper and lower limits that are not exceeded or fallen short of when generating a positive or negative bolus can be specified for the conductivity. The limits can be specified such that there is no burden on the patient.

In the embodiment described above, the first clearance value is determined on the basis of the change in a physical or chemical property of the dialysis fluid. An alternative embodiment for determining the blood flow in the vascular access Qa provides for the estimation of the first clearance value D instead of the measurement. FIG. 10 shows the individual method steps of the alternative embodiment. Method steps 100 and 101, and 103 to 108 correspond to method steps 100, 101, and 105 to 110 of the first embodiment (FIG. 9). In this regard, reference is made to the description of the first embodiment for determining the first clearance value. Instead of method steps 102, 103 and 104 of the first embodiment, the first clearance value D is estimated in the second embodiment (step 102). The computing and evaluating unit 23 can determine an estimated value for the first clearance value according to the method described in EP 1 698 360 B1 or calculate said value according to the equations published in Sargent, A. & Gotch, F., ‘Principles and biophysics of Dialysis’ in ‘Replacement of renewal function by dialysis’, 4th ED.

A second aspect of the invention provides that the distance between the first size (A) of the first bolus and a reference value is greater than the distance between the second size (A′) of the second bolus and a reference value. According to the second aspect, a reference value is defined, the size of the at least one bolus for determining the first haemodialysis parameter and the size of the at least one bolus for determining the second haemodialysis parameter being related to the reference value. In the present embodiment, this reference value is the noise in the system, the distance from the reference value being the signal-to-noise ratio. For an accurate measurement, the largest possible signal-to-noise ratio is sought, which increases with the size of the bolus, but which also increases the burden on the patient. The individual method steps of the second aspect of the invention correspond to those of the first aspect, the dialysis device for determining the first haemodialysis parameter, in particular the blood flow in the vascular access Qa, specifying a bolus that has a greater signal-to-noise ratio than the bolus for determining the second haemodialysis parameter, in particular the dialysis dose.

1. A dialysis device configured for connecting a dialyser that is divided by a semipermeable membrane into a first compartment, which is part of an extracorporeal blood circuit, and a second compartment, which is part of a dialysis fluid system of the dialysis machine, comprising
a device for determining at least one first and second haemodialysis parameter, the device for determining a haemodialysis parameter comprising:
a device for generating a change over time in a physical or chemical parameter of a dialysis fluid in a dialysis fluid feed line to the dialyser in the dialysis fluid system in the form of a bolus,
a device for detecting a change over time in the physical or chemical characteristic of the dialysis fluid attributable to the bolus in a dialysis liquid discharge line from the dialyser in the dialysis fluid system,
a computing and evaluating unit cooperating with the device for generating a change over time in a physical or chemical parameter and the device for detecting a change over time in a physical or chemical parameter that is configured such that, on the basis of a change in the physical or chemical parameter, at least one value for the clearance of the dialysis treatment is determined, and, on the basis of the at least one value for the clearance, at least one haemodialysis parameter is determined, wherein
the device for generating a change in a physical or chemical parameter is designed such that, to determine a first haemodialysis parameter, at least one bolus for determining the first haemodialysis parameter is generated having a first size, the computing and evaluating unit being configured such that, on the basis of the change in the physical or chemical parameter in the form of at least one bolus for determining the first haemodialysis parameter, at least one value for the clearance of the dialysis treatment is determined, on the basis of which value or values the first haemodialysis parameter is determined, and
in that, to determine a second haemodialysis parameter, at least one bolus for determining the second haemodialysis parameter is generated having a second size, the computing and evaluating unit being configured such that, on the basis of the change in the physical or chemical parameter in the form of at least one bolus for determining the second haemodialysis parameter, at least one value for the clearance of the dialysis treatment is determined, on the basis of which value or values the second haemodialysis parameter is determined,
the first size being larger than the second size or the distance between the first sizeand a reference value being greater than the distance between the second size and a reference value.

2. The dialysis device according to claim 1, wherein the at least one bolus for determining the first haemodialysis parameter is characterised by a first area, and the at least one bolus for determining the second haemodialysis parameter is characterised by a second area, the first area content of the first area being greater than the second area content of the second area.

3. The dialysis device according to claim 1, wherein the first haemodialysis parameter is a blood flow in the vascular access or a recirculation flow in a vascular system.

4. The dialysis device according to claim 3, wherein the dialysis device is configured for connection to an extracorporeal blood circuit that comprises a first bloodline and a second bloodline that is to be connected to an inlet or outlet of the first compartment of the dialyser and
in that for determining the blood flow in the vascular access, the device for generating a change in a physical or chemical parameter is designed such that, for a first dialysis condition, a first bolus having the first area contentand, for a second dialysis condition, a second bolus having the first area content is generated, the computing and evaluating unit being configured such that, on the basis of the change in the physical or chemical parameter in the form of the first bolus having the first area content under the first dialysis condition, a first value for the clearance is determined and, on the basis of the change in the physical or chemical parameter in the form of the second bolus under the second dialysis condition, a second value for the clearance is determined, on the basis of which values the blood flow in the vascular access is determined, or
to determine the blood flow in the vascular access, the device for generating a change in a physical or chemical parameter is designed such that a bolus having the first area content is generated for a second dialysis condition, the computing and evaluation unit being configured such that, on the basis of an estimate of the clearance for the first dialysis condition, a first value for the clearance is determined and, on the basis of the change in the physical or chemical parameter in the form of the bolus having the first area content under the second dialysis condition, a second value for clearance is determined, on the basis of which values the blood flow in the vascular access is determined, wherein,
under the first dialysis condition, the first bloodline conveys blood from a downstream portion of a vascular access of the patient and the second bloodline conveys blood towards an upstream portion of the vascular access and, under the second dialysis condition, the first bloodline conveys blood from an upstream part of the vascular access and the second bloodline conveys blood towards a downstream portion of the vascular access.

5. The dialysis device according to claim 1, wherein the second haemodialysis parameter is the dialysis dose Kt/V, K being the clearance, t the dialysis time and V the urea distribution volume.

6. The dialysis device according to claim 5, wherein for determining the dialysis dose Kt/V, the device for generating a change in a physical or chemical parameter is designed such that a bolus is generated having the second area content, the computing and evaluating unit be configured such that, on the basis of the change in the physical or chemical parameter in the form of the bolus having the second area content, a value for the clearance is determined, on the basis of which value the dialysis dose Kt/V is determined.

7. The dialysis apparatus according to claim 1, wherein a controlling and computing unit cooperating with the device for generating a change in a physical or chemical parameter and the device for detecting a change over time in a physical or chemical parameter is provided, which controlling and computing unit is configured such that the generation and detection of the change in the physical or chemical parameter is started during the blood treatment to determine the first or second haemodialysis parameter.

8. The dialysis machine according to claim 7,
wherein the controlling and computing unit is configured such that, to determine the first haemodialysis parameter, the generation and detection of the change in the physical or chemical parameter is started only once or only twice during the blood treatment and/or
in that, to determine the second haemodialysis parameter, the generation and detection of the change in the physical or chemical parameter is started multiple times during the blood treatment.

9. The dialysis apparatus according to claim 4, wherein, to determine the first haemodialysis parameter, the device for generating a change in a physical or chemical parameter is designed such that the substance concentration of a substance in the dialysis fluid, is increased to determine the first and second values of the clearance under the first and second dialysis conditions or the value of the clearance under the second dialysis condition or decreased to determine the first and second values of the clearance under the first and second dialysis conditions or the value of the clearance under the second dialysis condition and/or
to determine the second haemodialysis parameter, the device for generating a change in a physical or chemical parameter is designed such that the substance concentration of a substance in the dialysis fluid, is increased for a previous measurement and decreased for a subsequent measurement or decreased for a previous measurement and increased for a subsequent measurement.

10. A dialysis system comprising the dialysis device according to claim 1, and comprising a dialyser that is divided by a semipermeable membrane into a first compartment, which is part of an extracorporeal blood circuit, and a second compartment, which is part of a dialysis fluid system of the dialysis apparatus, and comprising an extracorporeal blood circuit that comprises a first bloodline and a second bloodline that is connected to an inlet or outlet of the first compartment of the dialyser.

11. A method for determining at least one first and second haemodialysis parameter during a dialysis treatment with a dialysis device comprising the following method steps:
Generating at least one change over time in a physical or chemical parameter of a dialysis fluid in a dialysis fluid feed line to a dialyser in a dialysis fluid system in the form of a bolus,
Detecting at least one change over time in the physical or chemical parameter of the dialysis fluid attributable to the at least one bolus in a dialysis fluid discharge line from the dialyser in the dialysis fluid system,
Determining at least one value for the clearance of the dialysis treatment on the basis of the at least one change in the physical or chemical parameter in the form of a bolus, and determining at least one haemodialysis parameter on the basis of the at least one value for the clearance, wherein,
to determine a first haemodialysis parameter, at least one bolus for determining the first haemodialysis parameter is generated having a first size, at least one value for the clearance of the dialysis treatment being determined on the basis of the change in the physical or chemical parameter in the form of at least one bolus for determining the first haemodialysis parameter, on the basis of which value the first haemodialysis parameter is determined, and
in that, to determine a second haemodialysis parameter, at least one bolus for determining the second haemodialysis parameter is generated having a second size, at least one value for the clearance of the dialysis treatment being determined on the basis of the change in the physical or chemical parameter in the form of at least one bolus for determining the second haemodialysis parameter, on the basis of which value the second haemodialysis parameter is determined,
the first size being larger than the second size or the distance between the first size and a reference value being greater than the distance between the second size and a reference value.

12. The method according to claim 11, wherein the at least one bolus for determining the first haemodialysis parameter is characterised by a first area and the at least one bolus for determining the second haemodialysis parameter is characterised by a second area, the first area content of the first area being greater than the second area content of the second area.

13. The method according to claim 11, wherein the first haemodialysis parameter is a blood flow in the vascular access or a recirculation flow in a vascular system.

14. The method according to claim 13, wherein,
to determine the blood flow in the vascular access, a first bolus having the first area content is generated for a first dialysis condition and a second bolus having the first area content is generated for a second dialysis condition, a first value for the clearance being determined on the basis of the change in the physical or chemical parameter in the form of the first bolus having the first area content under the first dialysis condition and a second value for the clearance being determined on the basis of the change in the physical or chemical parameter in the form of the second bolus under the second dialysis condition, on the basis of which values the blood flow in the vascular access is determined, or
to determine the blood flow in the vascular access for a second dialysis condition, a bolus having the first area content is generated, a first value for the clearance being determined on the basis of an estimate of the clearance for the first dialysis condition and a second value for the clearance being determined on the basis of the change in the physical or chemical parameter in the form of the bolus having the first area content under the second dialysis condition, on the basis of which values the blood flow in the vascular access is determined, wherein,
under the first dialysis condition, the first bloodline conveys blood from a downstream portion of a patient’s vascular access and the second bloodline conveys blood towards an upstream portion of the vascular access and, under the second dialysis condition, the first bloodline conveys blood from an upstream portion of the vascular access and the second bloodline conveys blood towards a downstream portion of the vascular access.

15. The method according to claim 12, wherein the second haemodialysis parameter is the dialysis dose Kt/V, where K is the clearance, t is the dialysis time and V is the urea distribution volume, and in that, to determine the dialysis dose Kt/V, a bolus having the second area content is generated, a value for the clearance being determined on the basis of the change in the physical or chemical parameter in the form of the bolus having the second area content, on the basis of which value the dialysis dose Kt/V is determined.

16. A computer program product that comprises commands that, when the program is executed by a computer, cause the computer to carry out a method according to claim 11 for determining at least one first and second haemodialysis parameter during a dialysis treatment with a dialysis device.

17. The dialysis apparatus according to claim 4, wherein, to determine the first haemodialysis parameter, the device for generating a change in a physical or chemical parameter is designed such that the substance concentration of a substance in the dialysis fluid, that is a Na concentration, is increased to determine the first and second values of the clearance under the first and second dialysis conditions or the value of the clearance under the second dialysis condition or decreased to determine the first and second values of the clearance under the first and second dialysis conditions or the value of the clearance under the second dialysis condition and/or
to determine the second haemodialysis parameter, the device for generating a change in a physical or chemical parameter is designed such that the substance concentration of a substance in the dialysis fluid, that is a Na concentration, is increased for a previous measurement and decreased for a subsequent measurement or decreased for a previous measurement and increased for a subsequent measurement.
